(19) United States
(12) Patent Application Publication
(10) Pub. No.: US 2023/0159871 A1
(43) Pub. Date: May 25, 2023

(54) BIOMIMETIC HEART TISSUE CULTURE SYSTEM

(71) Applicant: University of Louisville Research Foundation, Inc., Louisville, KY (US)

(72) Inventors: Guruprasad GIRIDHARAN, Louisville, KY (US); Moustafa MEKI, Prospect, KY (US); Tamer M.A. MOHAMED, Goshen, KY (US)

(21) Appl. No.: 17/997,764

(22) PCT Filed: May 14, 2021
(86) PCT No.: PCT/US2021/032372

§ 371 Date: Nov. 2, 2022

Related U.S. Application Data
(60) Provisional application No. 63/024,795, filed on May 14, 2020.

(51) Int. Cl. C12M 3/00; C12N 5/077; C12M 1/42; C12M 1/36; C12M 1/32; G01N 33/50

(52) U.S. Cl. CPC C12M 21/08; C12N 5/0657; C12M 35/02; C12M 35/04; C12M 41/48; C12M 23/12; G01N 33/5014

(57) ABSTRACT

An in vitro three-dimensional multicellular system for maintaining tissue such as heart tissue (e.g. human or other animal heart slices) under physiological conditions is provided. Heart tissue that is cultured using the system remains full viable and functional for at least 6 days. The system is thus suitable to continuously monitor the effects of drugs (e.g. cardiotoxicity) during in vitro testing.

### CROSS-REFERENCE TO RELATED APPLICATIONS

This application claims benefit U.S. provisional patent application assigned U.S. Ser. No. 63/024,795, filed May 14, 2020.

### BACKGROUND OF THE INVENTION

### Field of the Invention

The invention generally relates to improved systems and methods of culturing heart tissue in vitro. In particular, the invention provides an in vitro 3-dimensional culture system that mimics physiological conditions to maintain heart tissue viability for a period exceeding 24 hours, permitting reliable evaluation of drug candidates.

### Description of Related Art

Drug induced cardiotoxicity is a major cause of market withdrawal and failure of new drug candidates (2). In the last decade of the 20th century, eight non-cardiovascular approved drugs, including Cisapride and Rofecoxib, were withdrawn from clinical use because they induced cardiac arrythmia (9), resulting in sudden death. This is despite significant preclinical and clinical testing to evaluate the safety of these drugs. In addition, several approved cancer therapies (while in many cases effective) can lead to several cardiotoxic effects including cardiomyopathy and arrhythmias. For example, both traditional (e.g. anthracyclines and radiation) and targeted (e.g. trastuzumab) breast cancer therapies can result in cardiovascular (CV) complications in a subset of patients (10). Less clear are the CV effects of newer agents, including newer Her2 and PI3K inhibitors, especially when therapies are used in combination. Therefore, there is a growing need for reliable preclinical screening strategies for CV toxicities associated with emerging cancer therapies prior to human clinical trials. Unfortunately, the current testing methods of cellular in vitro assays and in vivo animal models poorly predict human cardiac liabilities, posing a multi-billion-dollar burden on the pharmaceutical industry. Hence, there is a worldwide unmet medical need for better approaches to identify drug toxicity before undertaking costly and time consuming ‘first in human’ trials.

The recent move towards the use of human induced pluripotent stem cell-derived cardiomyocytes (hiPSC-CMs) in cardiotoxicity and drug efficacy testing has provided a partial solution to address this issue; however, the immature nature of the hiPSC-CMs and loss of tissue integrity compared to multicellular heart tissue are major limitations of this technology. However, the use of this technology has been severely limited by the short period of viability in culture, which does not extend beyond 24 hours using the most robust protocols reported till early 2019 (4-6) due to multiple factors including not incorporating physiologic mechanical loading, air-liquid interface, and use of a simple medium that does not support the demands of the cardiac tissue.

Human cardiac slices have emerged as a promising model of the human heart for scientific research and drug testing. By retaining the normal tissue architecture, a multi-cell type environment, and the native extracellular matrix, human cardiac slices can faithfully replicate organ-level adult cardiac physiology, including replication of the human myocardium's physiological and pathological conditions. However, while viable slices of human myocardium approach these demands, their rapid degeneration in tissue culture precludes long-term experimentation (>24 hours) while acute cardiotoxicity testing requires several days for continuous exposure. This rapid degeneration is due to multiple factors including not incorporating physiologic mechanical loading, air-liquid interface, and use of a simple medium that does not support the demands of the cardiac tissue. Thus, the lack of availability of culture systems for human heart tissue that is functionally and structurally viable for more than 24 hours is a limiting factor in reliable cardiotoxicity testing.

There is an urgent need for culture systems that can accurately replicate the in vivo environment seen in the human heart. Furthermore, such a culture system with prolonged heart tissue viability could be used for mechanistic understanding of cardiovascular disease and therapies.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof. Evaluation of drugs using cultures of human heart slices is a promising model to simulate intact human myocardium. The present disclosure provides a complete 3D multicellular system that mimics human heart tissue under physiological and/or pathological conditions, both functionally and structurally, providing a reliable system for culturing human heart tissue e.g. for testing drug toxicity and other purposes. The novel biomimetic culture system advantageously maintains full viability and functionality of both human and pig heart slices (for example, 300 μm thickness) for at least 6 days in culture, thereby permitting continuous monitoring to detect any subtle cardiotoxic effects and closing the gap between preclinical and clinical testing results.

It is an object of this invention to provide an apparatus comprising: a base comprising a fluid chamber which is open at a top surface of the base; a middle section positioned on the base, wherein the middle section comprises: i) a media chamber which is open at a bottom surface of the middle chamber, wherein the fluid chamber of the base and the media chamber of the middle section are separated by a flexible membrane; and ii) one or more tissue culture wells recessed into a top surface of the middle section, wherein the one or more tissue culture wells is in fluid communication with the media chamber; an upper section comprising at least one pair of open slots which extend from a top surface of the upper section and through the upper section and which open at a bottom surface of the upper section; a top comprising a top upper surface and a top bottom surface, wherein the top comprises at least two electrodes extending from the top upper surface, through the top and out from the top bottom surface, wherein the at least two electrodes are configured to be received by the at least one pair of open slots in the upper section; one or more inserts positioned within the one or more tissue culture wells, wherein each of the one or more inserts comprises two posts having upper post surfaces configured to receive and support a tissue sample; and an electrode circuit board configured to electrically engage with at least two electrodes at the top upper surface.

Also provided in an in vitro culture system, comprising the apparatus of claim 1 and one or more of the following: an electrical stimulator configured to provide an electrical current to the at least two electrodes; a pneumatic driver configured to exert mechanical pressure across the flexible membrane; a reservoir for culture medium; and a pumping system configured to circulate the culture medium from the reservoir and through the media chamber. In some aspects, the in vitro culture system further comprising a force transducer. In additional aspects, the tissue sample is heart tissue and the system further comprises: an ECG monitor configured to receive electrical signals from the heart tissue, and a strain gauge configured to receive contractile function signals from the heart tissue. In further aspects, the electrical stimulator delivers a current at a frequency of 1.2 Hz (72 cycles per minute). In yet further aspects, the mechanical pressure is cyclical and cycles from 0-125 mmHg In yet further aspects, the current frequency can be in the range of 40-250 cycles per minute and the mechanical pressure can vary from 0-300 mmHg In other aspects, the in vitro culture system further comprises a computerized monitoring system. In additional aspects, the computerized monitoring system is a continuous monitoring system. In additional aspects, the computerized monitoring system monitors one or both of a contractile function signal and an electrocardiogram (ECG) signal in each cell culture well.

Also provided is a method of culturing heart tissue in vitro, comprising culturing a heart tissue sample in the in vitro culture system as described herein. In some aspects, the heart tissue sample is a human heart tissue sample. In further aspects, the step of culturing is performed for at least 6 days.

Also provided is a method of testing heart toxicity of a drug, comprising, culturing a heart tissue sample in the in vitro culture system as described herein; during the step of culturing, contacting the heart tissue sample with the drug, and after the step of contacting, measuring at least one of a contractile function signal and an electrocardiogram (ECG) of the heart tissue sample, wherein an aberration in the contractile function signal and/or the ECG indicates that the drug is toxic to the heart. In some aspects, the steps of culturing, contacting and measuring are performed for at least 6 days. In further aspects, the heart tissue sample is a human heart tissue sample.

### BRIEF DESCRIPTION OF THE DRAWINGS

FIG. 1. Exemplary working system with showing different components.

FIG. 2. Exemplary components of a 6-well device.

FIG. 3. Exemplary electrical components of a 6-well device; the electrode circuit board provides electrical pacing connections.

FIG. 4. Cutout view of an exemplary device showing various components.

FIGS. 5A and B. A, posts used to hold tissue slices inside culture wells; B, view of culture wells with discs in place.

FIG. 6. Schematic drawing of an exemplary device.

FIG. 7. Schematic drawing of a cardiac tissue slice positioned in an exemplary continuous monitoring system for contractile function and ECG recording in each tissue culture well.

FIG. 8. Pressure and voltage stimulation recording during the culture shows the synchronization of pressure changes with the electrical stimulation signal.

FIG. 9A-E. Schematic representations of exemplary parts of the device. A, top with electrodes; B, upper section with slots to accommodate electrodes; C, middle section with tissue culture wells; D, base with fluid chamber; E, circuit board; F, membrane; G, insert.

FIG. 10. An exemplary system.

### DETAILED DESCRIPTION

This disclosure describes a tissue biomimetic culture device and a system comprising the device. In some aspects, the system is adapted to culture heart tissue samples, such as heart tissue slices of e.g. pig or human hearts. Cardiac physiology and pathology is influenced by cross talk between the various cell types within the myocardium; thus, the present heart slice model system, which advantageously uses e.g. 3D heart tissue rather than single cardiac cells in culture or rodent heart tissue, facilitates understanding the mechanisms of and evaluating efficacy and safety of new therapeutics. For example, the safety and efficacy of agents (either new or old) designed to treat heart failure can be tested and evaluated using the system, since the culture system faithfully replicates the physiological environment of the in vivo heart. These conditions are reproduced by using physiological mechanical stimulation and a culture media that is advantageous to cardiac tissue slices. By using this physiologically relevant approach, the viable culture time of heart tissue in the culture system has been prolonged to more than 6 days. The prolonged culture of viable cardiac tissue permits preclinical testing of e.g. new, experimental heart failure therapies on human heart tissue at early stages of drug development, such as before clinical trials, and also provides a valuable tool to study human heart biology.

### The Device

The present culture system (device, apparatus, etc.) comprises multiple sections or parts. Those of skill in the art will recognize that although the “parts” of the present device are described separately, some of the parts may also be manufactured as a single piece. All possible combinations of i) parts that are manufactured separately and then assembled and affixed together (e.g. by gluing, soldering, etc.); and ii) parts, two or more of which are manufactured as a single component to be assembled with other components, are encompassed herein.

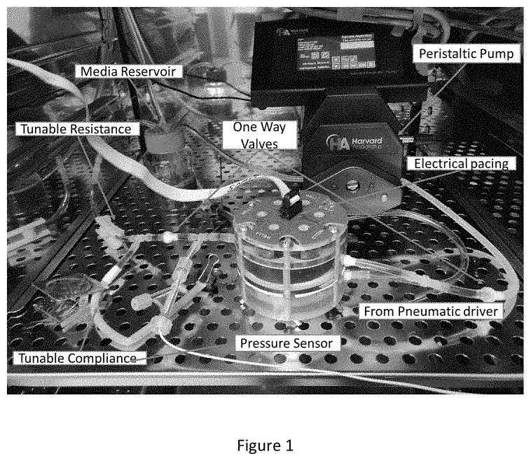

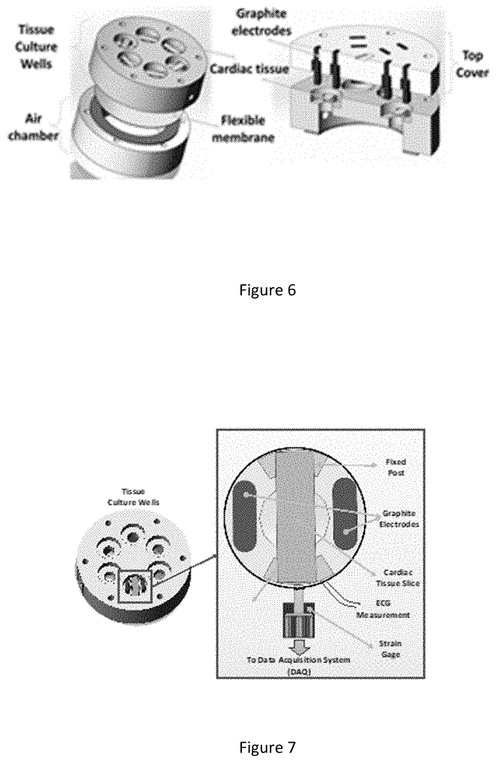

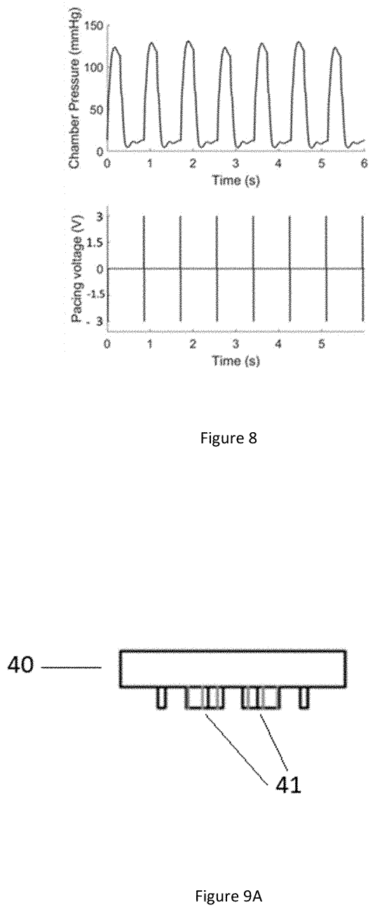

An exemplary culture system is depicted in FIGS. 1-7. As can be seen, the culture system described herein comprises at least the following components:

Base with Fluid Chamber

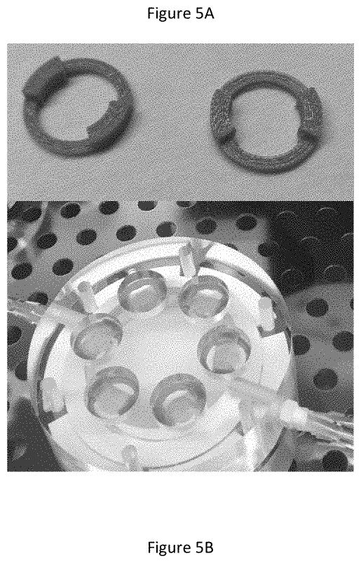

The base of the system (i.e. the bottom portion that contacts a surface on which the device is placed when in use, such as a lab bench, table or shelf of an incubator) is shown in FIG. 4 and comprises a solid bottom surface and walls extending vertically upward from the bottom surface. The walls define an open chamber (an “air or fluid chamber”) so that a top portion of an upper surface of the base is open, i.e. it is not closed unless occluded by the placement or attachment of another component of the device. The shape of the base may be any that is suitable for inclusion in the device, e.g. substantially cylindrical, square, rectangular, irregular, etc. as long as the base fits tightly and operably with the other device components. The base may also include one or more inlet and/or outlet channels in the wall of the base (if the base is cylindrical) or in one or more walls of the base (if the base is e.g. rectangular). In some aspects, the base and the chamber within the base are both substantially cylindrical. As the name implies, during use of the device, the chamber is generally filled with a “fluid” and the one or more channels may function as an inlet and/or outlet for circulating fluid into and out of the air chamber. Any type of gas or liquid may function as a “fluid”, examples of which include but are not limited to: air, helium, nitrogen, water, oxygen, carbon-dioxide, and saline.

The fluid chamber is connected to a pulsatile pneumatic driver. The pneumatic driver fills and empties the chamber and is used to actuate the flexible membrane. The membrane does not act as a diffusion barrier, but as an actuator which deflects (distends) under pressure to create pulsatile pressure and flow in the culture chamber, which is described below. This pulsation stretches the tissue that is being cultured, similar to how such tissue stretches during a heartbeat. The membrane is not required to allow for any gas diffusion or liquid diffusion.

### Middle Section

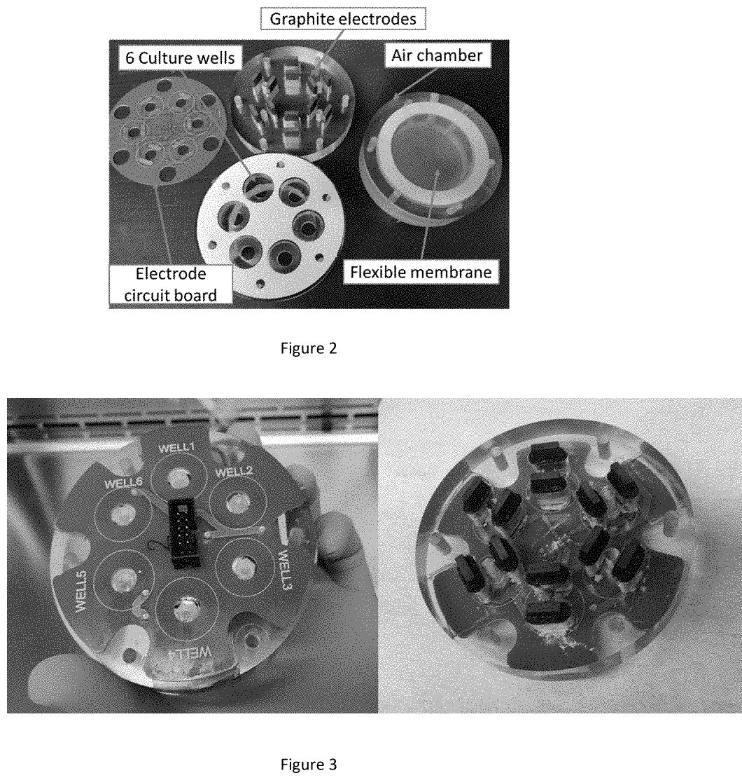

Also shown in FIGS. 2, 4 and 5B, is a mid-portion (middle section) of the device. The mid-portion comprises a top surface and walls extending vertically down therefrom to define a media chamber, the bottom of which is open. When assembled in the device, the mid-portion sits atop the base so that the open section of the bottom of the media chamber aligns with (is directly on) the open section of the top of the base portion, albeit indirectly since the two are separated by a flexible membrane, which is described below. The chamber in the mid-portion functions as and is designed to contain media and comprises at least one inlet (for fresh media to enter the chamber) and at least one outlet (for media to exit the chamber). During use, media typically flows through (into and out of) the chamber continuously and so mimics the constant flow of blood around the heart in vivo.

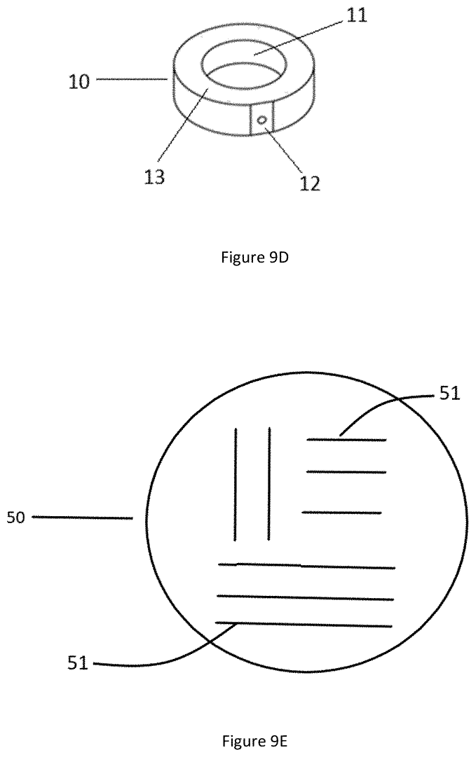

The flow of media in and out of the chamber provides perfusion and a good supply of oxygenated media to the cultured tissue. In another aspect of the invention, oxygenated media can be held in the middle chamber and mechanical stimulation can be provided to the tissue without flow in or out of the middle chamber, for short periods of time (few hours). The mimicking of the heart condition is done through the pulsatile element of the flow which is done through the flexible silicone membrane which will distend under pressure to create pulsatile pressure and pulsatile flow, similar to that in the normal heart, as shown in FIG. 8.

In addition, the mid-portion comprises at least one tissue culture well. Generally, a plurality of wells are included, e.g. from about 2-108, including all integers in between, such as about 6, 12, 18, 24, 30, 36, 42, 48, 54, 60, 66, 72, 78, 84, 90, 96, 102, or 108 or more. In some aspects, the number of wells is a multiple of 6. The wells are recessed into the top surface of the mid-portion and are open at the top surface of the mid-portion. A bottom surface of each well comprises a centrally located open channels that extends vertically downward from the bottom surface of the tissue culture well to the media chamber below so that the wells are in fluid communication with the media chamber. The diameter of the open channel is less than that of the bottom surface or the well. Thus, the bottom surface of each well also comprises a “rim” around the open channel which provides support for an insert (described below). Tissue samples positioned in the culture wells are continuously exposed to the media in the media chamber via the open channel so that nutrients can be supplied to the samples and waste products can be removed.

The culture wells are generally cylindrical in form (although other shapes may be adopted). The volume of a culture well generally ranges from about 0.05 in to about 2 in, including all integers and decimal fractions to the hundredth place in between and/or the dimensions of a tissue culture well are generally about 1 in×1 in (1 in diameter); however, other larger or smaller well sizes are encompassed, so long as the wells are capable of receiving a tissue sample as described herein. The channels that connect the tissue culture wells to the media chamber may also be cylindrical in shape, although other shapes are not precluded. The dimensions of the channels are generally 0.1 to about 1 inch, such as about 0.1, 0.2, 0.3, 0.4, 0.5, 0.6, 0.7, 0.8, 0.9 or about 1.0. The dimensions must be such that some exchange of media occurs between the tissue culture wells and the media chamber.

When in use, each tissue culture well generally contains an insert that fits snugly within the bottom of the well and against the rim and walls of the well. The insert comprises a central opening and so surrounds but does not block or occlude the central open channel of the well. The insert comprises “posts” or “support surfaces” that protrude upward. In some aspects, at least two posts are present on opposite sides of the insert, their function being to provide, at their top surface, a support for a tissue sample that is positioned thereon. The top surface of the posts is sufficient to support the tissue sample but does not occlude the culture well. Rather, the area between and adjacent to the posts is open so that media in the well comes into direct contact with and surrounds the tissue sample suspended across and supported by the top surface of the posts.

### Flexible Membrane

As noted above, the two aligned chambers (air chamber and media chamber) are separated by a flexible, liquid impermeable membrane (FIG. 4). The membrane is flexible so that it distends in response to the air pressure inside the air chamber. When the membrane is moving, it will push the media above it, thereby creating pressure on the fluid side, which in turn distends the tissue. The use of pneumatic actuation allows for fast response and no contact with the sterile culture media. Materials that may be used to produce the membrane include but are not limited to: any biocompatible, flexible polymer (e.g. silicone, Polydimethylsiloxane (PDMS), latex rubber, Thermoplastic elastomers), and the like.

The flexible membrane is generally produced separately from the other system components and then attached during assembly of the device. Those of skill in the art will recognize that attachment of the flexible membrane may involve the use of gaskets and machine screws to seal the points of contact between the membrane and the base below and the middle section above. Also, the membrane may be permanently bonded to the gasket or bottom chamber via oxygen plasma bonding, sonic welding or other techniques. In some aspects, the membrane is sandwiched between two circular gaskets above and below it, and all sections of the device are then held together tightly by the aid of 6 radially positioned machine screws.

### Upper Section

The device also comprises an upper section that sits atop the middle section in an assembled device (FIGS. 2, 3 and 4). The upper section comprises at least two channels (“electrode channels”) or passages that extend vertically and completely through the upper section (from a top surface through to a bottom surface, being open at both surfaces. Each pair of two channels is configured to receive a pair of electrodes. When assembled in a device, the channels extend from the upper surface of the upper section, through the upper section and into the culture wells, i.e. they open into the culture wells. Thus, when placed in the electrode channels, electrodes extend into the media in the culture wells. At least one pair (i.e. two) electrodes are present for each culture well.

It is noted that the middle section and the lower section are generally produced separately and joined e.g. via a gasket, and machine screws and nuts, as described above for the connection of the flexible membrane. It is noted that when using gaskets, gluing may not be necessary. The flexible gaskets are held tightly in between the rigid plastic parts (i.e. the culture chamber and the air chamber to create a tight seal.

Alternatively, these two sections may be produced as one unit.

The lower section can be of any suitable shape and is generally shaped so as to “fit” the portion(s) of the device with which it engages (is directly attached to), i.e. the middle section. In some aspects, the upper section is substantially cylindrical.

The dimensions of the lower section, e.g. for a 6-well device, are typically about 02.25 in diameter and the dimensions of the channels are typically about 00.38 in diameter. However, those of skill in the art will recognize that the dimensions can be scaled up or down. The size of all components, including wells and the size of the overall chamber, can vary as needed or desired, to suit an intended use.

### Top Section

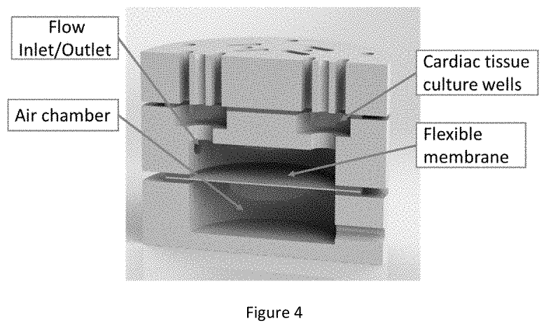

The device also comprises a top section that fits atop the upper section (see FIGS. 2 and 3). The top section has a top surface and a bottom surface and at least one pair of electrodes protrudes from the bottom surface of the top section. The electrodes are configured so as to extend into the electrode channels of the upper section i.e. the electrodes fit into or engage with the electrode channels in a manner so that that one end (a lower end) of each electrode enters the culture well and makes contact with the media therein.

The electrodes that are utilized in the device are typically rectangular and made of is molded graphite and of sufficient width to ensure a uniform electric field is applied to the tissue. The electrodes can be made of platinum or other conductive material. However, any suitable type of electrode may be used.

The upper end of each electrode is sufficiently exposed at the top surface of the upper section so as to electrically connect to (make an electrical connection with) a circuit board that sits directly on the top surface of the top section (see FIGS. 2 and 3).

The top section can be of any suitable shape and is generally shaped so as to “fit” the portions of the device with which it engages. In some aspects, the top section is substantially cylindrical.

The dimensions of the top section (e.g. of a 6-well device) are typically about Φ1.875 in×0.6 in.

As discussed above, an electrical circuit board is attached to the top section of the device, e.g. at the top or on the side. In some aspects, the circuit board sits atop the top section (is attached to the top surface of the top section). In this case, the circuit board is shaped so as to fit and substantially cover the top surface of the top section. The purpose of the electrical circuit board is to provide an electrical interface between the electrodes and an external source of electricity so as to electrically stimulate the tissue slices in culture using predefined field stimulation. This allows the used to control the electric pulses to the heart tissue.

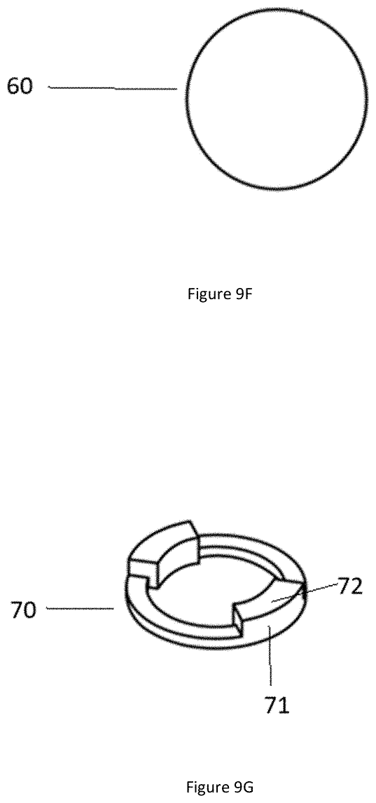

Accordingly, FIG. 9D shows a top view of base 10 comprising fluid chamber 11 and air inlet 12. Also shown is top surface 13. The flexible membrane rests on top surface 13 when the device is fully assembled.

Also shown in FIG. 9C is a top view of middle section 20 comprising culture wells 21. Rim 22 of culture well 21 receives an insert which supports a tissue sample Channels 23 extend downward from culture wells 21 and open into media chamber that is built into the bottom of middle section 20 (not shown). Media inlet 22 is shown and a similar opening is present e.g. on the opposite side of the middle portion and is not shown.

Also depicted in FIG. 9B is a top view of upper section 30. Upper section 30 comprises a plurality of pairs of channels 31, each pair being configured to receive two electrodes (from top section 40) when the device is fully assembled.

FIG. 9A depicts a side view of top section 40 of the device. As can be seen, pairs of electrodes 41 extend the bottom surface of top section 40. Each pair of electrodes 41 fits within a pair of channels 31 of upper section 30 when the device is fully assembled.

FIG. 9E shows a schematic representation of a top view of electrode circuit board 50, which is positioned on a top surface of top section 40 when the device is fully assembled. Exemplary generic circuit board components 51 are shown. In some aspects, holes are included in the top cover and are positioned at the center of each well (not shown). These are threaded holes can be used to de-air the fluid side when the device is being primed with media.

FIG. 9F depicts flexible membrane 60 which, as described above, sits between base 10 and middle section 20 when the device is fully assembled.

FIG. 9G shows an insert 70 which sits in a culture well 24. Posts 71 extend upward from the insert and serve to support a tissue sample. The two upper surfaces 72 of post 71 receive and supports a tissue sample and suspends the tissue sample across the insert. The central open channel permits full fluid communication between a culture well 24 (where the insert is placed) and the media chamber of the middle section 20, as described above.

### System

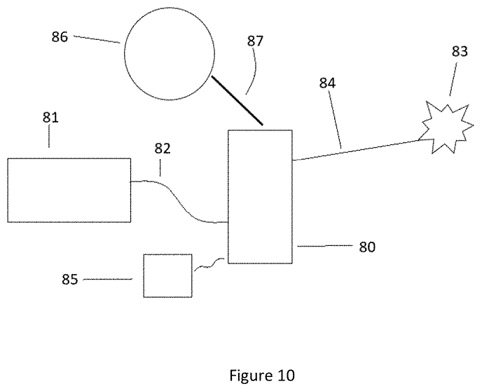

Also provided herein are systems which include at least one device as described above. An exemplary system is shown schematically in FIG. 10 and includes but is not limited to:

A device 80.

A media reservoir 81 configured to provide fresh medium. The medium reservoir may be connected to the device via, for example, suitable conduits such as a system of tubing 82 and valves (not shown) to control the flow into and out of the media chamber in the middle section. In addition, the media reservoir may be connected to a pumping system configured to circulate the culture medium from the reservoir, into the media chamber and out of the media chamber at a suitable flow rate to maintain tissue viability, such as about ˜80-115 ml/min. The pumping system is tuned along with the flow valves and pneumatic driver to achieve the target physiological pressure waveform morphology.

At least one power source 83 configured to provide electrical energy to one or more components of the system via one or more electrical connections 84. For example, the power source may be or may power an electrical stimulator configured to provide an electrical current to the two electrodes of the pair or pairs of electrodes present in the device.

A compressor and/or pneumatic driver 85 configured to ultimately exert mechanical pressure across the flexible membrane, and to provide and maintain a suitable flow of fluid to the air chamber in the base of device 80, under a suitable amount of pressure. In particular, the flow of air is cyclical/periodic, ranging from about 0 mm Hg to about 300 mm Hg with a periodicity of about 40-250 cycles per minute to provide accurate replication of pressures and pressure gradients within the heart in vivo under normal, exercise, and cardiac dysfunction physiologies. In general, a rate of about 72 cycles per minute (1.2 Hz) and about 40-125 cycles per minute simulates a normal heart rate, and values outside these parameters simulate e.g. hypotension, hypertension, intense exercise, etc. For example, a normal resting heart rate for adults typically ranges from about 60 to 100 beats per minute. Generally, a lower heart rate at rest implies more efficient heart function and better cardiovascular fitness. For example, a well-trained athlete might have a normal resting heart rate closer to 40 beats per minute. For example, an irregular heartbeat (arrhythmia), a low heartbeat (bradycardia, e.g. below 60 beats per minute), and/or a high heartbeat rate (tachycardia e.g. above 100 beats per minute) can be replicated. The air pressure, flow valves, and pumping system can be adjusted to replicate various physiological (e.g., exercise) and pathological (e.g., pressure or volume overload) conditions. In another aspect of the invention, the membrane can be directly actuated mechanically.

A computer system 86 which includes electrical connections 87 to the electrical components of device 80 and may also include one or more of: a data acquisition card to collect the pressure sensor data, processor for processing data from the device and synchronize the electrical stimulation with the cyclical pressure, and a display for displaying data, electrode array that can be lowered on the surface of the tissue to map electrical activity, and other sensors.

### Manufacturing

One or more of the various parts of the device may be manufactured by any suitable method, e.g. by molding, 3D printing, machining, laser cutting, etc.

The materials that are used to produce the parts of the device include but are not limited to: various solid plastics and resins and metals, which may be transparent or opaque, biocompatible flexible elastomers and rubbers, etc.

### Operation and Methods of Use

The disclosed culture system is designed to culture tissue in vitro for a prolonged period of time, e.g. at least about 1-12 days or longer, such as for about 1, 2, 3, 4, 5, 6, 7, 8, 9, 10, 11, 12 days, or even for up to e.g. 60 days, such as for about 10, 20, 30, 40, 50 or 60 days. In some aspects, the tissue remains viable for at least about 6 days.

The media and the conditions that are used are generally specific for the type of tissue, for example, heart tissue such as slices of heart tissue. In this aspect, the culture system maintains simultaneous mechanical and electrical stimulation with continuous monitoring to detect any subtle cardiotoxic effects in heart slices during culture, e.g. with new drugs. In some aspects, the heart slices are of human origin. In other aspects, the heart slices are of porcine, bovine, ovine, canine, murine, rat or other mammalian origin.

The media that is used to culture heart tissue is as follows: M199 culture media, which is enriched with Insulin-Transferrin-Selenium (ITS), Vascular Endothelial Growth Factor (VEGF), Fibroblast Growth Factor (FGF), Fetal Bovine Serum (FBS). Th culture media is changed once every day and the fresh culture media is oxygenated. Continuous oxygenation of media can also be accomplished while the media is being recirculated. Other media that provides required nutrients can also be used. The media can be altered to mimic certain physiologic conditions, e.g. hypoxia, high levels of certain metals/toxins, etc.

In practice, a 3D heart tissue sample is obtained (harvested) from a donor, for example, from a biopsy, from a cadaver or an animal that has been sacrificed. 3D printed tissues or cardiac cells attached to a flexible membrane, engineered heart tissue, 3D lab grown tissue, etc. can also be used. As used herein, a “3D heart tissue sample” or “slice” refers to a sample of tissue having a thickness of from about 100 to 500 μm (e.g. about 100, 150, 200, 250, 300, 350, 400, 450, or 500 μm), such as about 300 μm. The other dimensions of the sample are generally about 1 cm×0.5 cm. However, are no particular limits to the tissue size itself (length×width) as long as the culture wells can accommodate the sample. The sample shape may be any convenient or suitable shape, such as substantially rectangular, substantially circular, irregular, etc. The sample shape may be modified to fit the post surfaces on which it is placed, or vice versa, i.e. the post surfaces of a device may be designed and manufactured to accommodate a particular size and shape of tissue samples that are to be cultured in the device. Generally, harvested tissue is kept in a suitable solution, such as a Tyrode solution with BDM, on ice to reduce its activity. The tissue is considered fresh for 2-3 hours after harvest of the organ.

The harvested tissue sample is placed in the system, e.g. so as to be supported on and across the upper surfaces of the posts within a cell culture well, as described above. Generally, the tissue must be placed in the device immediately after harvest (e.g. within 2-3 hours), unless it is preserved some manner that permits transit from the location of harvest to the site of testing. Media is generally already circulating into the system and through the culture wells.

In a typical scenario, a heart is harvested and kept in a cardioplegic solution in an ice bath until it is delivered to a user of the device/system. The heart slice harvesting is done by the user and the ventricular block from which slices are cut is kept in Tyrode solution with BDM for at least one hour before being placed in the bath to remove any traces of the cardioplegic solution.

Once the tissue sample(s) are positioned in the culture wells, the device is sealed and placed in a standard incubator with controlled temperature condition and CO₂ supply in vivo physiological conditions are replicated by i) applying a suitable electrical pulse to the sample to mimic the electrical signals of the heart; and ii) applying suitable pressure through the flexible membrane to simulate rhythmic contractions of the heart.

Once culture conditions are established, one or more agents to be tested are added to the media of test samples in at least one device. Control samples, in at least one different device, do not receive the one or more agents. Data obtained from each device that is used is collected during the test and is transmitted electrically e.g. to a computerized processing system, to be analyzed. The data can advantageously be collected and monitored continuously in real time. Data that is obtained includes but is not limited to: contractile function, Pressure, Shear stress, electrophysiological properties, strain, and electrical signal.

### Uses

The human-relevant models disclosed herein are used to, for example: accurately test the toxicity and/or efficacy of drugs, including drugs that are intended to treat cardiac conditions (for which e.g. efficacy, dosing and toxicity can be determined); and drugs intended to treat other conditions e.g. to determine whether a drug or a combination of drugs has side effects that are toxic to heart tissue. In addition, the effects of combinations of drugs of any type can be assessed, to determine e.g. favorable or unfavorable interactions.

Drugs that are tested may be new (e.g. not yet approved by the FDA) or may be known drugs for which further testing would be beneficial, e.g. to determine interactions with other agents.

In addition, the substances that are tested include agents other than “drugs” per se. For example, nutraceuticals, various chemicals that are released into the environment and/or that are produced during manufacturing, metals, synthetic monomers and/or polymers used in manufacturing, breakdown products of e.g. plastics, various gases, the effects of radiation, environmental toxins, air pollution substances, and waste water, etc. The effects of any agent of combination of agents that can be introduced into the system, e.g. into the culture medium, may be tested.

In addition, the devices and systems described herein are used to: assess both structural and functional abnormalities of cardiac tissue including diseased states; avoid false-positive findings because of animal-specific data obtained using other systems; to identify delayed responses, reveal patient-specific susceptibilities, and detect/predict off-target effects.

Further, the external media circuit allows for accurate replication of pressures and pressure gradients experienced in the heart and can simulate different pathological conditions such as hypoxia, hypertension, tachycardia, and bradycardia.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1. Development of Biomimetic Heart Slice Culture System Combining the Electrical and Mechanical Stimulation

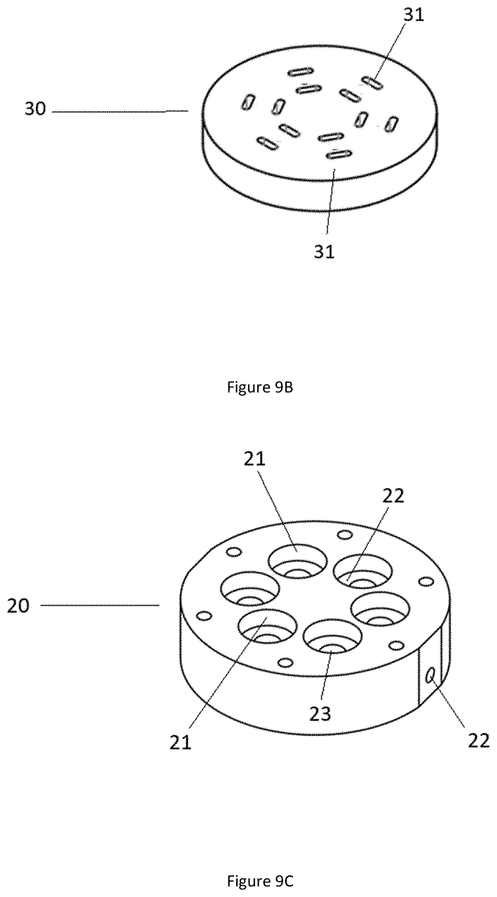

A prototype 6-chamber tissue culture system for continuous monitoring of contractile function and ECG recording in each tissue culture well was created using rapid prototyping techniques (FIG. 1). FIGS. 2-6 show various view of exemplary components of the device, as described in the Figure legends. FIG. 7 depicts a schematic drawing of a cardiac tissue slice positioned in the device.

The tissue culture chamber was fabricated using a two-step process of crosslinking prepolymer and spinning on a silicon wafer to obtain membranes that were molded and manufactured to have six chambers bound to the silicon wafer exposed to oxygen plasma in a reactive ion etcher. Each chamber was provided two graphite electrodes for electrical stimulation and connection to a C-Pace stimulator (Ionoptix, Inc). This assembly was laid on a standard 6 cell culture air chamber and driven by a programmable pneumatic driver (LB Engineering, Germany) The air chamber and tissue chamber are separated by a flexible PDMS membrane. This composite was sandwiched between two polycarbonate plates. The top polycarbonate piece contains an inlet and outlet channel micro machined using an end-mill cutter and containing connections for inlet and outlet tubing. A peristaltic pump is used to circulate the media in the tissue chamber. Pressure waveform morphology was controlled using tunable resistance and compliance elements. The design of the setup enables constant circulation and oxygenation of the media.

In one aspect, the current biomimetic Cardiac Tissue Culture Model (CTCM) comprises two chambers separated by a flexible silicone membrane. The bottom chamber is connected to a programmable pneumatic driver which is used to provide mechanical stimulation to the tissue samples in the upper chamber and simulate the physiologic pressures inside the heart. The top chamber has 6 radially oriented wells. Each well houses one cardiac tissue slice (300 μm thickness) fixed on a post. Each tissue slice is placed between two graphite electrodes that provide electrical pacing with an electric field intensity of −3V/cm. The 6 wells are connected to an external mock circulation circuit comprised of tubing, resistance and compliance elements and a peristaltic pump.

FIG. 8 shows typical pressure and voltage stimulation recording during culturing. As can be seen, pressure changes are synchronized with the electrical stimulation signal. In particular, the 6-chamber design produced simultaneous mechanical loading (0-125 mmHg) and electrical stimulation. The incorporation of continuous contractility and electrophysiology monitoring of mechanical and electrical stimulation in the culture system extended the viability of the heart slices beyond day 6.

### Example 2

In some aspects, an electrode array is lowered onto the tissue to measure and record the electrical activity of the tissue. Multi axis tissue strain is measured using strain gages or using optical means to estimate the strain or characterize the stretch of the tissue. The membrane is directly actuated mechanically or the tissues are actuated/stretched mechanically.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. An apparatus comprising
a base comprising a fluid chamber which is open at a top surface of the base;
a middle section positioned on the base, wherein the middle section comprises:
i) a media chamber which is open at a bottom surface of the middle chamber,
wherein the fluid chamber of the base and the media chamber of the middle section are separated by a flexible membrane, and
ii) one or more tissue culture wells recessed into a top surface of the middle section, wherein the one or more tissue culture wells is in fluid communication with the media chamber;
an upper section comprising at least one pair of open slots which extend from a top surface of the upper section and through the upper section and which open at a bottom surface of the upper section;
a top comprising a top upper surface and a top bottom surface, wherein the top comprises at least two electrodes extending from the top upper surface, through the top and out from the top bottom surface, wherein the at least two electrodes are configured to be received by the at least one pair of open slots in the upper section;
one or more inserts positioned within the one or more tissue culture wells, wherein each of the one or more inserts comprises two posts having upper post surfaces configured to receive and support a tissue sample; and
an electrode circuit board configured to electrically engage with the at least two electrodes at the top upper surface.

2. An in vitro culture system, comprising
the apparatus of claim 1 and one or more of the following:
an electrical stimulator configured to provide an electrical current to the at least two electrodes;
a pneumatic driver configured to exert mechanical pressure across the flexible membrane;
a reservoir for culture medium; and
a pumping system configured to circulate the culture medium from the reservoir and through the media chamber.

3. The in vitro culture system of claim 2, further comprising a force transducer.

4. The in vitro culture system of claim 2, wherein the tissue sample is heart tissue and the system further comprises:
an ECG monitor configured to receive electrical signals from the heart tissue, and
a strain gauge configured to receive contractile function signals from the heart tissue.

5. The in vitro culture system of claim 2, wherein the electrical stimulator delivers a current frequency of 40-250 cycles per minute.

6. The in vitro culture system of claim 2, wherein the mechanical pressure is cyclical and cycles from 0-300 mmHg

7. The in vitro culture system of claim 2, further comprising a computerized monitoring system.

8. The in vitro culture system of claim 7, wherein the computerized monitoring system is a continuous monitoring system.

9. The in vitro culture system of claim 7, wherein the computerized monitoring system monitors one or both of a contractile function signal and an electrocardiogram (ECG) signal in each cell culture well.

10. The in vitro culture system of claim 2, wherein the current frequency is 72 cycles per minute and the mechanical pressure cycles from 0 to 125 mmHg to simulate normal physiology.

11. A method of culturing heart tissue in vitro, comprising
culturing a heart tissue sample in the in vitro culture system of claim 2.

12. The method of claim 11, wherein the heart tissue sample is a human heart tissue sample.

13. The method of claim 11, wherein the step of culturing is performed for at least 6 days.

14. A method of testing heart toxicity of a drug, comprising
culturing a heart tissue sample in the in vitro culture system of claim 2,
during the step of culturing, contacting the heart tissue sample with the drug, and
after the step of contacting, measuring at least one of a contractile function signal and an electrocardiogram (ECG) of the heart tissue sample,
wherein an aberration in the contractile function signal and/or the ECG indicates that the drug is toxic to the heart.

15. The method of claim 14, wherein the steps of culturing, contacting and measuring are performed for at least 6 days.

16. The method of claim 14, wherein the heart tissue sample is a human heart tissue sample.
